(19) United States
(12) Patent Application Publication
(10) Pub. No.: US 2026/0048082 A1
(43) Pub. Date: Feb. 19, 2026

(54) BIOPOLYMER-BASED HYDROGELS AND METHOD FOR ENTEROSORPTIVE REMOVAL OF TARGET MOLECULES

(71) Applicant: University of Kentucky Research Foundation, Lexington, KY (US)

(72) Inventors: Mei Li Weatherly, Lexington, KY (US); Ifrah Hammad, Lexington, KY (US); Sachin Sundar, Lexington, KY (US); James Zach Hilt, Lexington, KY (US)

(21) Appl. No.: 19/280,702

(22) Filed: Jul. 25, 2025

Related U.S. Application Data
(60) Provisional application No. 63/675,417, filed on Jul. 25, 2024.

(51) Int. Cl. A61K 33/44; A61K 9/00; A61K 9/06; A61K 47/36

(52) U.S. Cl. CPC A61K 33/44; A61K 9/0053; A61K 9/06; A61K 47/36

(57) ABSTRACT

A method for enterosorptive removal of a target molecule from a digestive tract of an individual, includes orally administering a biopolymer-based hydrogel adapted to bind and remove the target molecule from the digestive track without being metabolized or absorbed into systemic circulation. The biopolymer-based hydrogel may be an biopolymer-based hydrogel composite and the method further includes incorporating an additive into the biopolymer-based hydrogel selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

### RELATED APPLICATION

This application claims the benefit of U.S. Provisional Patent Application Ser. No. 63/675,417, filed on Jul. 25, 2024, the full disclosure of which is incorporated herein by reference.

### TECHNICAL FIELD

This document relates generally to the field of enterosorption and, more particularly to biopolymer-based hydrogels and related methods adapted for binding and removing harmful target molecules from the digestive tract of an individual without being metabolized or absorbed into the systemic circulation and thereby excreted unchanged with the absorbed target molecules.

### BACKGROUND

Per- and poly-fluoroalkyl substances (PFAS) pose documented and suspected health problems to humans, such as abnormal fetal development, increased risk of cancer, immunosuppression, and thyroid dysfunction. Due to prevalent concentrations of PFAS in local drinking water, various consumer products, and other commonplace items, removal of PFAS from the body is of interest to the EPA. Current PFAS treatment methods include anion exchange, reverse osmosis, and nanofiltration; however, none of these methods are suitable for in vivo removal of PFAS.

Synthetic food dyes, specifically FD&C food dyes, are a class of FDA-approved food dyes from non-naturally derived sources such as petroleum and coal tar. Currently, there are seven FD&C straight food dyes: Red #40, Red #3, Yellow #5, Yellow #6, Green #3, Blue #1, Blue #2. While research is inconclusive, the synthetic food dyes are suggested to cause multiple adverse health effects. For example, Red #40 is strongly suggested to be carcinogenic, Yellow #5 and Yellow #6 are linked to inducing allergenic reactions in individuals who have urticaria and other chronic allergy predispositions, and Blue #1 and Blue #2 are linked to attention deficit and other developmental-behavioral issues. Despite these adverse health effects, lobbyist support for synthetic food dyes remains strong, and although other countries have tentatively restricted or outright banned the use of FD&C dyes, the United States has yet to follow suit due to increasing corporate pressure to allow FD&C dyes due to the psychological propensity of vibrant coloring to induce hunger. While the FDA recently banned the use of Red #3, following many of its European predecessors, other petitions to ban synthetic food dyes have not been addressed.

Due to the multifaceted nature of PFAS and synthetic food dyes, outright bans and successful removal from mass consumption are difficult and tedious tasks hindered by lobbying, consumerism, and their substantial prevalence in consumables. As a result, an in vivo method for their removal from the body, one that directly mitigates the health impacts of their accumulation and presence, is needed. Enterosorption is defined as an oral adsorbent that is used to bind and remove contaminants from the digestive tract without being metabolized or absorbed into systemic circulation themselves, thus being excreted unchanged with the adsorbed contaminant. In literature, enterosorption has been used for the removal of mycotoxins, heavy metal toxins, and endo/exo-toxins.

Biopolymers, such as agarose, have been used in literature for controlled substance release and target drug delivery both in vitro and in vivo. Furthermore, agarose is “generally regarded as safe” by the FDA within their approved guidelines. The biopolymer-based hydrogels set forth in this document are useful in a method for the enterosorptive removal of target molecules, such as PFAS, dyes and synthetic food dyes thereby providing for the first time an effective biopolymer-based hydrogel system and method adapted for in vivo removal of these contaminants from the digestive tract.

### SUMMARY

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “an additive”, as used herein, may also refer to, and encompass, a plurality of additives.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10% of the stated numerical value.

In accordance with the purposes and benefits set forth herein, a method for enterosorptive removal of a target molecule from a digestive tract of an individual, comprises, consists of or consists essentially of orally administering a hydrogel or hydrogel composite adapted to bind and remove the target molecule from the digestive track without being metabolized or absorbed into systemic circulation. The hydrogel composite incorporates an additive that may be selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

Similarly, a method for enterosorptive removal of a target molecule from a digestive tract of an individual, comprises, consists of or consists essentially of orally administering a biopolymer-based hydrogel adapted to bind and remove the target molecule from the digestive track without being metabolized or absorbed into systemic circulation. In at least some embodiments of the method, the biopolymer-based hydrogel is a biopolymer-based hydrogel composite and the method further includes incorporating an additive into the biopolymer-based hydrogel. The additive may be selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

In at least some embodiments, the biopolymer-based hydrogel is a biopolymer-based hydrogel composite and the method further includes incorporating an additive into the biopolymer-based hydrogel selected from a list of additives consisting of an activated carbon, a powdered activated carbon, a granular activated carbon, an extruded activated carbon, an impregnated activated carbon, and combinations thereof. In at least some embodiments, the biopolymer-based hydrogel is an biopolymer-based hydrogel composite and the method further includes incorporating an additive into the biopolymer-based hydrogel selected from a list of polymers consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, and combinations thereof.

In some embodiments, the biopolymer-based hydrogel is a biopolymer-based hydrogel composite and the method further includes incorporating an additive into the biopolymer-based hydrogel selected from a list of polymers consisting of poly(lysine), poly(ethyleneimine), chitosan, cationic cellulose, poly(acrylic acid), poly(methacrylic acid), and combinations thereof. In some embodiments, the biopolymer-based hydrogel is an biopolymer-based hydrogel composite and the method further includes incorporating an additive into the biopolymer-based hydrogel selected from a list of additives consisting of a lectin, a DNA binding protein, an RNA binding protein, an albumin, a carbohydrate, and combinations thereof.

The method may be particularly useful by further including binding and removing:
(a) per-fluoroalkyl substances from the digestive tract;
(b) poly-fluoroalkyl substances from the digestive tract;
(c) dye substances from the digestive tract; and
(d) synthetic food dye from the digestive tract.

The method may further include administering the biopolymer-based hydrogel composite at a dosage rate of between 0.002 to 1.0 grams at a time interval of between every 4 hours and once a month. The method may further include incorporating a weight percentage of additive of between about 0.1% and 50% into the biopolymer-based hydrogel composite. In some embodiments, the method may further include incorporating a weight percentage of additive of between about 0.1% and 20% into the biopolymer-based hydrogel composite.

In accordance with an additional aspect, a hydrogel system comprises, consists of or consists essentially of a biopolymer-based hydrogel adapted for oral administration to an individual wherein the biopolymer-based hydrogel is further adapted to bind and remove a target molecule from the digestive track without being metabolized or absorbed into systemic circulation. In some embodiments, the biopolymer-based hydrogel is an biopolymer-based hydrogel composite incorporating an additive into the biopolymer-based hydrogel selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

In some embodiments, the biopolymer-based hydrogel is a biopolymer-based hydrogel composite and the method further includes incorporating an additive into the biopolymer-based hydrogel selected from a list of additives consisting of an activated carbon, a powdered activated carbon, a granular activated carbon, an extruded activated carbon, an impregnated activated carbon, and combinations thereof. In some embodiments, the biopolymer-based hydrogel is biopolymer-based hydrogel composite and the method further includes incorporating an additive into the biopolymer-based hydrogel selected from a list of polymers consisting of poly(styrene), poly(methyl methacrylate), poly(ethylene), polypropylene, poly(vinyl chloride), polytetrafluoromethylene, poly(dimethylsiloxane), polyester, polyurethane, poly(vinylidene fluoride), fluoropolymer, and combinations thereof.

In other embodiments, the biopolymer-based hydrogel is a biopolymer-based hydrogel composite and the method further includes incorporating an additive into the biopolymer-based hydrogel selected from a list of polymers consisting of poly(lysine), poly(ethyleneimine), chitosan, cationic cellulose, poly(acrylic acid), poly(methacrylic acid), and combinations thereof. In still other embodiments, the biopolymer-based hydrogel is a biopolymer-based hydrogel composite and the method further includes incorporating an additive into the biopolymer-based hydrogel selected from a list of additives consisting of a lectin, a DNA binding protein, an RNA binding protein, an albumin, a carbohydrate, and combinations thereof.

In at least some of the possible embodiments, the hydrogel system further includes a weight percentage of additive of between about 0.1% and 50% in the biopolymer-based hydrogel composite In other possible embodiments, the weight percentage of additive of between about 0.1% and 20% in the biopolymer-based hydrogel composite.

In the following description, there are shown and described several embodiments of the hydrogel system and method for enterosorptive removal of a target molecule. As it should be realized, the hydrogel system and method are capable of other, different embodiments and its several details are capable of modification in various, obvious aspects all without departing from the hydrogel system and method as set forth and described in the following claims. Accordingly, the drawing figures and descriptions should be regarded as illustrative in nature and not as restrictive.

### BRIEF DESCRIPTION OF THE DRAWING FIGURES

The accompanying figures incorporated herein and forming a part of the specification, illustrates certain aspects of the new and improved method and together with the description serves to explain certain principles thereof. A person of ordinary skill in the art will readily recognize from the following discussion that alternative embodiments of the method may be employed without departing from the principles described below.

FIG. 1 illustrates the nomenclature for the biopolymer-based hydrogels and hydrogel composites.

FIG. 2 is a schematic illustration of the of the procedure used to form agarose hydrogels and hydrogel composites.

FIG. 3 is a schematic illustration of the procedure used to determine hydrogel swelling characteristics at equilibrium.

FIG. 4 is a schematic illustration of the procedure used to determine hydrogel standard swelling characteristics at equilibrium.

FIG. 5 is a schematic illustration of the procedure using Beer's Law to create a relationship between absorbance and concentration (calibration).

FIG. 6 is a schematic illustration of the procedure used to determine the sorption capacity of agarose hydrogel and hydrogel composites.

FIG. 7 is a graph of a swelling study of freeze-dried agarose-only and agarose-powdered activated carbon (PAC) (n=3) after one week in deionized water and gastric acid. Gastric acid pH=1.2 (0.2% NaCl) (w/v) in hydrochloric acid 0.7%.

FIG. 8 is a graph illustrating the removal of metanil yellow by freeze-dried agarose and agarose-powdered activated carbon hydrogels at t=90 hours in a temperature controlled orbital shaker at 37° C. (n=3). Solution concentration: 40 ppm. Sorbent dosage: 1 mg/mL.

FIG. 9 is a graph illustrating removal of metanil yellow by freeze-dried agarose and agarose-powdered activated carbon hydrogels at t=24 hours in a temperature-controlled orbital shaker at 37° C. (n=3). Solution concentration: 40 ppm. Sorbent dosage: 1 mg/mL.

FIG. 10 is a graph illustrating removal of metanil yellow by freeze-dried agarose and agarose-powdered activated carbon hydrogels at t=2 weeks in a temperature-controlled orbital shaker at 37° C. (n=3). Solution concentration: 40 ppm. Sorbent dosage: 1 mg/mL.

Reference will now be made in detail to the present preferred embodiments of the method for enterosorptive removal of a target molecule.

### DETAILED DESCRIPTION

A method for enterosorptive removal of a target molecule from a digestive tract of an individual may be broadly described as including orally administering a hydrogel or hydrogel composite to the individual wherein the hydrogel or hydrogel composite is adapted to bind and remove the target molecule from the digestive track without being metabolized or absorbed into systemic circulation. In at least some embodiments, the hydrogel is a biopolymer-based hydrogel and the hydrogel composite is an biopolymer-based hydrogel composite. For purposes of this document, a “biopolymer-based hydrogel” refers to a hydrogel made from a biopolymer, that is processed into a crosslinked network. Biopolymers useful as a basis for the biopolymer-based hydrogels include, but are not necessarily limited to agarose, alginate, cellulose, starch, gelatin, collagen, polysaccharides, chitin, chitosan, hyaluronic acid, nucleic acid-based polymers, carrageenan, carbohydrates, and proteins.

The method may further include incorporating an additive into the hydrogel. The additive may be selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

The terminology “biological polymer” refers broadly to natural polymers produced by cells of living organisms, including plants, animals, bacteria and fungi. The three main classes of biological polymers include polynucleotides, polypeptides and polysaccharides. Ribonucleic acid (RNA) and deoxyribonucleic acid (DNA) are examples of long polymers of nucleotides. Polypeptides include proteins and shorter polymers of amino acids (e.g. collagen, actin and fibrin). Polysaccharides are linear or branched chains of sugar carbohydrates (e.g. starch, cellulose and alginate). Other examples of biological polymers include, but are not necessarily limited to natural rubber, suberin, lignin, cutin, cutan, melanin, and ployhydroxyalkanoates.

Useful additives include, but are not necessarily limited to: (a) activated carbon, a powdered activated carbon, a granular activated carbon, an extruded activated carbon (in its smallest size range of around or below 1.0 mm diameter), an impregnated activated carbon, (b) polymers consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, poly(lysine), poly(ethyleneimine), chitosan, cationic cellulose, poly(acrylic acid), poly(methacrylic acid), and (c) additives, such as, a lectin, a DNA binding protein, an RNA binding protein, an albumin, a carbohydrate. Any combination of the above listed additives may also be used.

The biopolymer-based hydrogels and biopolymer-based hydrogel composites are particularly useful for binding and removing per-fluoroalkyl substances, poly-fluoroalkyl substances, dyes and synthetic food dues, including Red #40, Red #3, Yellow #5, Yellow #6, Green #3, Blue #1, Blue #2 from the digestive tract of an individual. For purposes of this document, “individual” refers to humans, mammals, domestic farm animals, and other animals including a digestive tract.

The biopolymer-based hydrogel or biopolymer-based hydrogel composite may be administered to the individual in a pharmaceutically effective amount such as at a dosage rate of between 0.002 to 1.0 grams over a period of every four hours, up to and including every month. Alternative dosage rates include, but are not necessarily limited to: between 0.002 and 0.8 grams, between 0.002 and 0.7 grams, between 0.002 and 0.5 grams, between 0.002 and 0.3 grams, between. 002 and 0.2 grams, between 0.005 and 1 gram, between 0.005 and 0.8 grams, between 0.005 and 0.5 grams, between 0.1 and 1 gram, between 0.1 and 0.8 grams, between 0.1 and 0.5 grams, between 0.3 and 1 gram, between 0.3 and 0.8 grams, between 0.3 and 0.5 grams, between 0.5 and 1 gram, and between 0.5 and 0.8 grams. Alternative time periods for each dose may also vary to a period of between every four hours and every week, between every 8 hours and every month, between every eight hours and every week, between every 12 hours and every month, between every 12 hours and every week, between every day and every month, between every day and every week, and between every 24 hours and every 48 hours. The dosage for treatment with the biopolymer-based hydrogels and hydrogel composites depends on such factors as the age, weight and condition of the individual as well as the condition being treated. The compound may be administered before meals for maximum effect to be obtained.

The weight percentage of additive added to the biopolymer-based hydrogel composite may be between 0.1% and 50%. In other embodiments, the weight percentage of additive added to the biopolymer-based hydrogel composite may be between 0.1% and 20%. In still other embodiments, the weight percentage of additive added to the biopolymer-based hydrogel composite may be between 0.5% and 50%, between 0.5% and 20%, between 1.0% and 50%, between 1.0% and 20%, between 5% and 50%, between 5% and 20%, between 10% and 50%, between 10% and 20%, between 25% and 50%, between 25% and 40% and between 30% and 40%.

The biopolymer-based hydrogel or biopolymer-based hydrogel composite may be made by any appropriate method for making hydrogels and hydrogel composite known to those skilled in the art. Thus, the hydrogels may be synthesized, for example, by combining biopolymer-based powder and deionized (DI) water at various w/w concentrations in a borosilicate scintillation vial. For composite hydrogels, the desired additive or combination of additives is added to the deionized water alongside the biopolymer-based.

Solutions for hydrogel preparation may be placed in a hot water bath at a range of 85-95 degrees Celsius and stirred continuously with a magnetic stir bar until the powder is completely dissolved. Then, gels may be cast in a 48-well polystyrene plate in appropriate volume increments using an Eppendorf positive displacement pipette or other appropriate means known to those skilled in the art. A uniform metallic cylindrical cutting tool may be used to extract the hydrogels and hydrogel composites. All gels may then be washed in DI water. The gels may then be freeze-dried to preserve expansibility.

Typically, the biopolymer-based hydrogel or biopolymer-based hydrogel composite is prepared as a cylinder plug within a microplate well, but it should be appreciated that it may be cast into almost any shape. These are the bulk structures as prepared. These may be dried by freeze drying, vacuum drying, convective drying or other means known in the art and then crushed and ground into a powder using various techniques known to those skilled in the art to produce powders of desired particle size and size distributions. In the dry powder form, these biopolymer-based hydrogels and hydrogel composites may be added to various types of formulations, known to those skilled in the art, to produce tablets, capsules, gummies and the like for oral administration to an individual. In one possible embodiment, it is as simple as adding the dried hydrogel powder directly to the capsule and then sealing the capsule.

If desired, the dry powder form of the biopolymer-based hydrogels and hydrogel composites may be combined with any useful excipients of a type known in the art. Useful excipients include, but are not limited to, known binders, diluents, fillers, glidants, antioxidants, preservatives, sweeteners, stabilizing agents, coating agents and flavoring agents. Suitable excipients are substances that do not react with the hydrogel powder and any composite additives. Thus, the tablet or capsule may contain, for example, gum, starch, gelatin and/or a buffer.

### EXPERIMENTAL

### Materials

Agarose, Type 1 (CAS: 9012-36-6), Indigo carmine (CAS: 860-22-0), Fluorescein (CAS: 518-47-8), Metanil Yellow (CAS: 587-98-4), Tartrazine (CAS: 1934-21-0) were purchased from Sigma-Aldrich (Milwaukee, WI, USA, 53209). Sodium Montmorillonite Clay (CAS: 1318-93-0) was used as received. NovaSil (CAS: 1302-78-9) was purchased from BASF Nutrition. Powdered Activated Carbon, Norit GSX, steam activated, acid washed (CAS: 7440-44-0) was purchased from Thermo Fischer Scientific (Haverhill, MA, USA). Simulated Gastric Fluid (without Pepsin) (CAS: 7647-01-0, 7647-14-5) and Simulated Intestinal Fluid (without Pancreatin), USP XXII Formulation (CAS: 7778-77-0, 1310-73-2) were purchased from Ricca Chemical (Batesville, Indiana, USA, 47006). Dulbecco's Phosphate Buffered Saline (DPBS) 1× Sterile Solution (CAS: 7647-14-5, 7447-40-7, 7558-79-4, 7778-77-0, 7732-18-5) was purchased from Research Products International (Mount Prospect, Illinois, USA, 60056). All chemical components were used as received.

### Agarose Hydrogel and Hydrogel Composite Formation

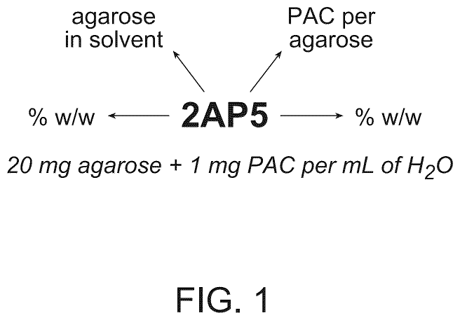

Agarose hydrogels were synthesized by combining agarose powder and deionized (DI) water at various w/w concentrations in a borosilicate scintillation vial of either 20 mL or 40 mL volume. For composite hydrogels, PAC was added alongside agarose. Compositions of agarose hydrogels and hydrogel composites are outlined in Table 1. FIG. 1 is a nomenclature description for the agarose-based hydrogel composite systems.

TABLE 1
Compositions of Agarose Hydrogels and Hydrogel Composites expressed
in mass per mL of water, rounded to the nearest tenth to represent
maximum precision of weigh scale. “AP” represents agarose
with powdered activated carbon as its additive (PAC) and “AC”
represents agarose with clay (either sodium montmorillonite clay or NovaSil).

| Sample Name (wt. % + | Agarose weight (mg) per mL of | Additive (mg) per mL of |
|---|---|---|
| additive(s)) | water | water |
| 2AP0 | 20 | — |
| 3AP0 | 30 | — |
| 4AP0 | 40 | — |
| 2AP5 | 20 | 1 |
| 3AP5 | 30 | 1.5 |
| 4AP5 | 40 | 2 |
| 2AP25 | 20 | 5 |
| 3AP25 | 30 | 7.5 |
| 4AP25 | 40 | 10 |
| 2AC5 | 20 | 1 |
| 3AC5 | 30 | 1.5 |
| 4AC5 | 40 | 2 |
| 2AC25 | 20 | 5 |
| 3AC25 | 30 | 7.5 |
| 4AC25 | 40 | 10 |

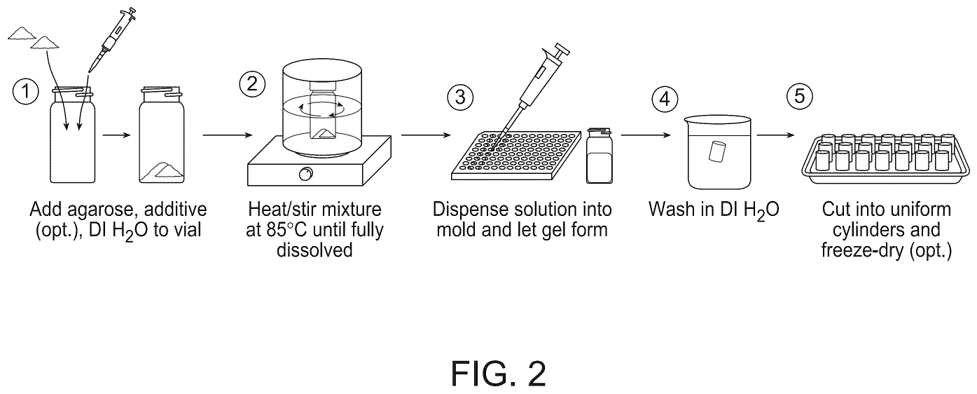

Solutions were placed in a hot water bath at a range of 85-95 degrees Celsius and stirred continuously with a magnetic stir bar until the powder was completely dissolved. Then, gels were cast in a 48-well polystyrene plate in 1 mL increments using an Eppendorf positive displacement pipette. A uniform metallic cylindrical cutting tool was used to extract hydrogels and hydrogel composites. All gels were washed 3× times in DI water. Gels were freeze-dried to preserve expansibility. A summarized schematic of the hydrogel formation procedure is shown in FIG. 2.

### Agarose Hydrogel and Hydrogel Composite Characterization

For both agarose hydrogels and hydrogel composites, Fourier Transform Infrared (FTIR) spectra were used to determine product functionality, which was used to ensure that agarose was not chemically modified during the formation process in hydrogel and hydrogel composites. FTIR spectra were obtained using the 7000e FTIR spectrometer (Varian Inc., Palo Alto, CA, USA). For analysis of dried hydrogels, the dried hydrogels were placed directly onto the crystal without further mechanical or chemical manipulation. Agarose hydrogels and hydrogel composites were analyzed for functional group retention to determine product functionality as well as to confirm that no unexpected reactions took place during the formation of agarose hydrogels (AG) and the agarose-PAC hydrogel composites (AP, AG+PAC).

To determine the incorporation of PAC into the agarose-PAC hydrogel composites, thermogravimetric analysis (TGA) was performed using Q600 SDT DSC-TGA (TA Instruments, New Castle, DE, USA) under nitrogen gas. The temperature procedure was as follows: ramp temperature from 110° C. at a rate of 20° C./min, hold isothermally for 10 minutes to stabilize, ramp temperature to 600° C. at a rate of 10° C./min, then hold isothermally for 10 minutes before cooling. Since the degradation of PAC—3550° C.—is higher than the degradation point of agarose—87° C.—and the maximum temperature is less than the degradation point of PAC but higher than the degradation point of agarose, the mass remaining of the hydrogel composite should quantify the PAC that is incorporated into the hydrogel network. TGA was performed on agarose hydrogels without PAC and then performed on hydrogels with PAC. Finally, the weight percents remaining of both systems were found, which was used to determine PAC incorporation, as demonstrated in Equation 1.

Equation 1: PAC Incorporation (%), Calculated by Subtracting the Agarose Hydrogel Final Weight % from the Agarose-PAC Composite Hydrogel Final Weight %.

$$wt {\%}_{final , AP} - wt {\%}_{final , AG} PAC \% \qquad ( 1 )$$

### Aqueous Swelling Studies of Agarose Hydrogels and Hydrogel Hybrids

To determine whether agarose polymer formed hydrogels, swelling behavior was examined through swelling studies. Equilibrium swelling studies were conducted using wet-to-dry (inverse) swelling as well as dry-to-wet (standard) swelling. To limit thermal impacts on agarose hydrogel mesh network composition, all swelling studies were conducted isothermally at room temperature (20-23° C.). Swelling was quantified through gravimetric comparison, as outlined in Equation 2.

Equation 2: Swelling Ratio (q) Using Swollen Hydrogel Mass (mₛ) and Dried Hydrogel Mass (m_{d})

$$q = \frac{{m}_{s}}{{m}_{d}} \qquad ( 2 )$$

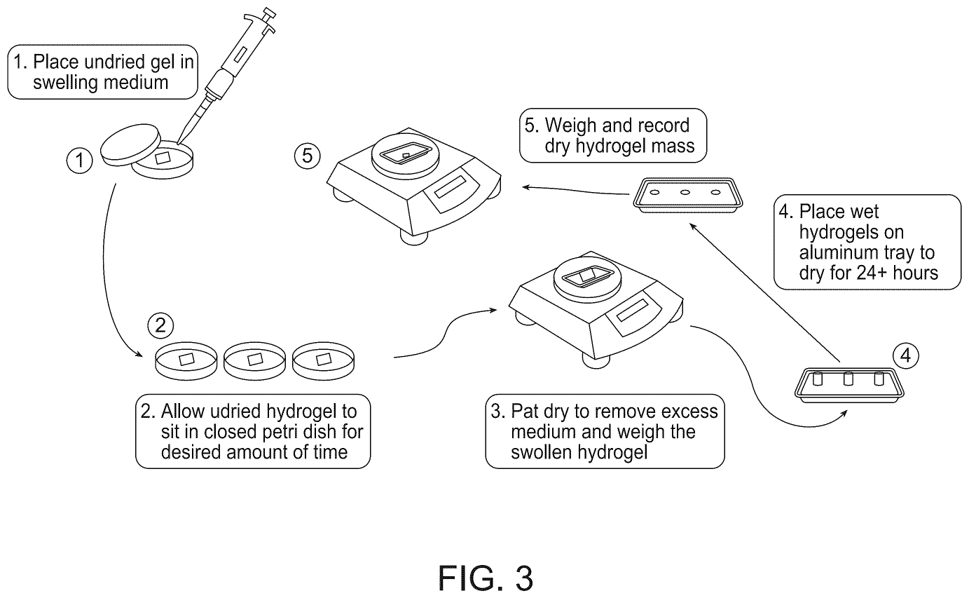

For inverse swelling studies, the newly formed, un-dried agarose hydrogels were submerged in deionized water (DI water) for at least 24 hours to reach equilibrium swelling. The surface of the swollen hydrogel was patted dry using Kimwipe tissues to remove excess DI water and weighed to find the equilibrium swollen weight of the agarose hydrogel. Afterward, swollen hydrogels were placed on aluminum trays to dry for at least 24 hours at room temperature (25° C.). Finally, the dried agarose hydrogel was weighed. All experiments were repeated in triplicate. Schematic is shown in FIG. 3.

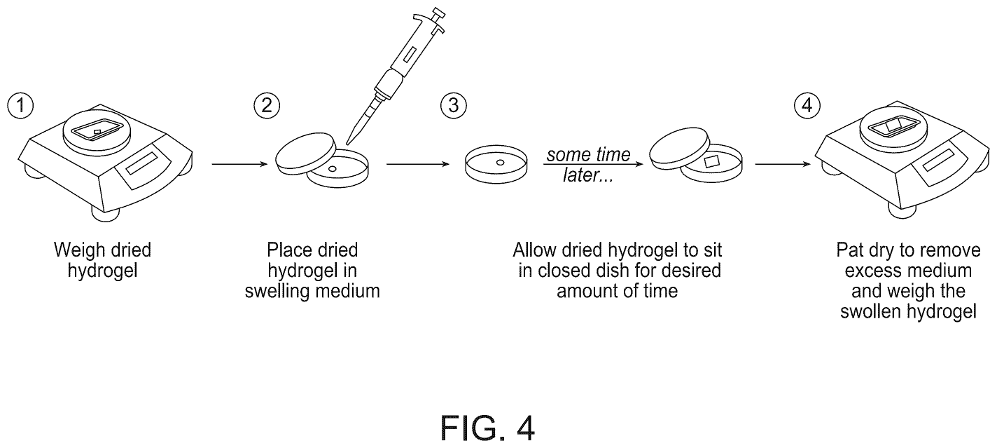

For standard swelling studies, dried hydrogels were weighed and then submerged in aqueous solution (simulated gastric acid, simulated intestinal fluid, DPBS, or DI water) for at least 24 hours to reach equilibrium swelling. Aqueous solutions were chosen to model the environment of the digestive tract, which an enterosorbent will need to traverse unencumbered and unmetabolized. The surface of the swollen hydrogel was patted dry using Kimwipe tissues to remove excess solution and weighed to find the equilibrium swollen weight of the agarose hydrogel. All experiments were completed in triplicate. Schematic is shown in FIG. 4.

### Sorption Study Calibration

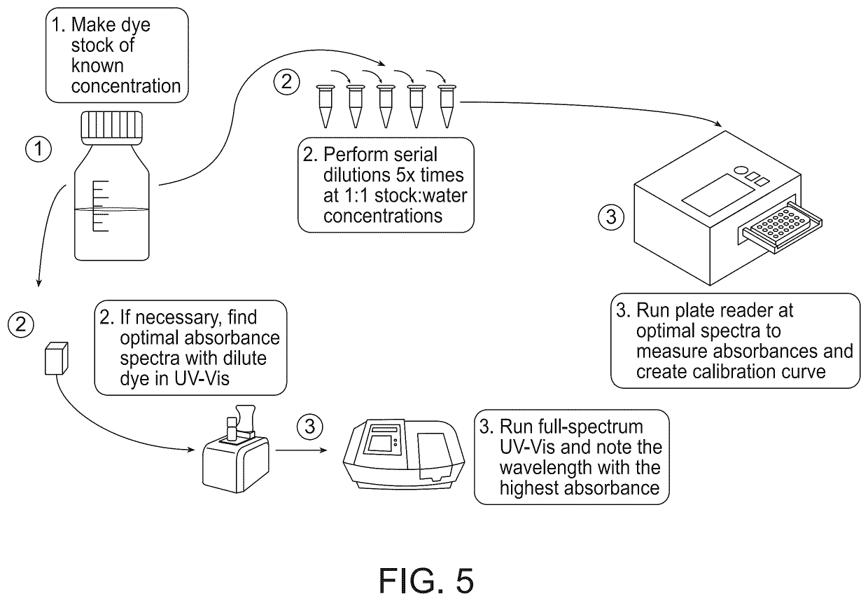

Dyes—fluorescein, indigo carmine, and metanil yellow—were selected based on functionality, molecular weight, and literature supporting similar behavior to PFAS in anionic exchange resins. To determine a relationship between absorbance and concentration of dye, a calibration curve was created using a microplate reader at maximum dye absorbance wavelength, which was determined to be 490 nm⁻¹ for fluorescein, 610 nm⁻¹ for indigo carmine, and 435 nm⁻¹ for metanil yellow through full-spectra Ultraviolet-visible spectroscopy, which was conducted with the Cary 60 UV-Vis Spectrophotometer (Agilent). A stock solution of a known concentration of dye was diluted with DI water in a 1:1 serial dilution to obtain 5× concentrations. The stock concentration was the same concentration as the sorption solution concentration. Schematic is shown in FIG. 5.

A microplate reader was then used to determine the absorbance values for each of the known concentrations. Beer-Lambert law establishes the linearity of absorbance with respect to concentration; however, this relationship is only valid for absorbances up to 1, which limits the concentration range of the calibration curve. Beer-Lambert Law is outlined in Equation 3.

Equation 3: Beer-Lambert Law. Absorbance (A) is Linearly Related to Concentration (C) Through an Absorption Constant (εl) where ε is the Proportionality Constant, and l is the Path Length (cm).

$$A = ϵ1 * C \qquad ( 3 )$$

### Sorption Study Procedure

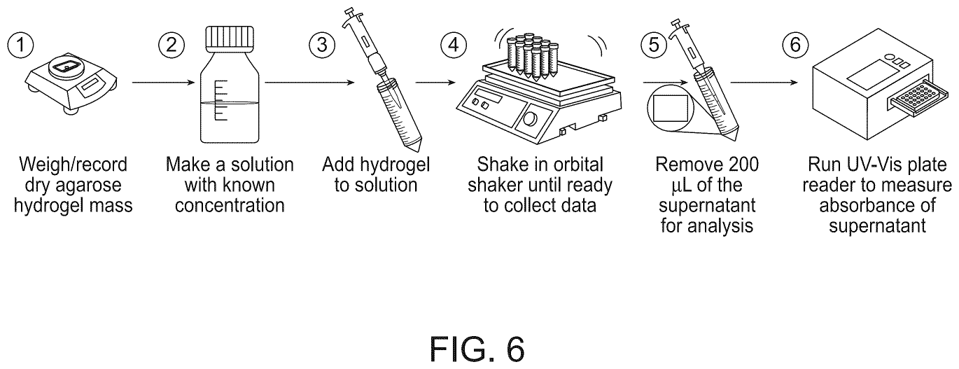

Dried agarose hydrogels of various agarose concentrations (cut to uniform volume prior to drying) were placed in Eppendorf polypropylene conical tubes. Tubes were agitated in an orbital shaker at 200 rpm until the desired time point was reached. 200 μL of supernatant was then removed and placed inside a 96-well microplate to be used in the microplate reader at the same absorbance as the calibration curve (490 nm-1 for fluorescein, 610 nm⁻¹ for indigo carmine, 435 nm⁻¹ for metanil yellow). All samples were collected in triplicate. Sorption studies were conducted isothermally at room temperature (22° C.). Data was quantified through removal efficiency (%), defined in Equation 4. The schematic is shown in FIG. 6.

Equation 4: Removal Efficiency, Expressed in Terms of Percent, Based on Initial Concentration (C₀) and Concentration at Time of Measurement (Cₜ).

$$Removal Efficiency ( \% ) = \frac{{C}_{0} - {C}_{t}}{{C}_{0}} * 100 \% \qquad ( 4 )$$

### Results and Discussion

Previously formed agarose-based hydrogel systems with powdered activated carbon incorporated into the hydrogel matrix were used.

### Swelling Study

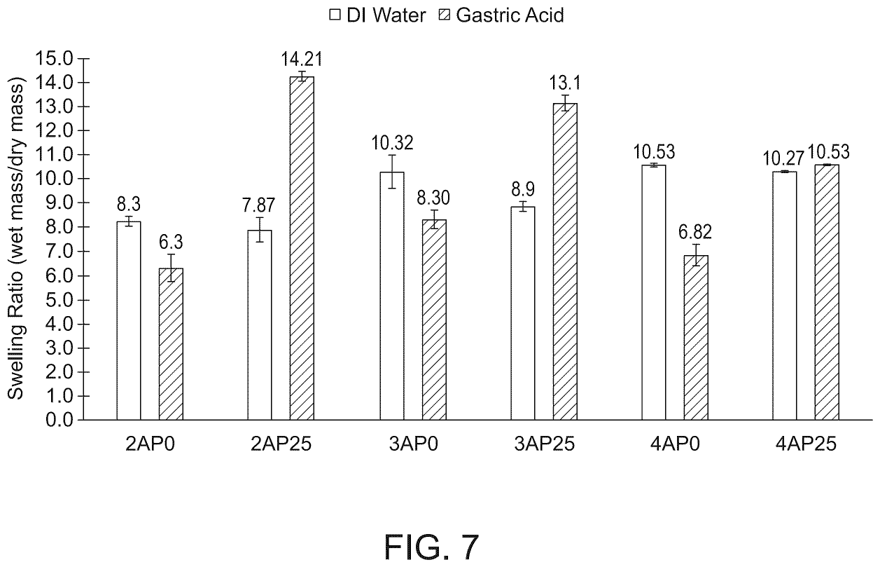

To determine hydrogel viability in acidic environments and ensure preserved expansibility, a swelling study using various agarose-PAC hydrogel composites was conducted on freeze-dried hydrogels. The results of the swelling study are outlined below. High degrees of swelling are present in all environments; however, the presence of activated carbon seems to increase the propensity of hydrogels to swell in gastric acid compared to their agarose-only counterparts. Conversely, the agarose-only counterparts have more of a propensity to swell in DI water than their agarose-PAC counterparts. See FIG. 7.

### Sorption Studies

To determine the removal efficiency of the freeze-dried agarose-based hydrogels, sorption studies were conducted with model dyes that exhibit similar behavior in anion exchange resins to PFOS (perfluorooctane sulfonic acid). Metanil yellow was specifically chosen due to its resistance to aqueous degradation over more extended periods of time (>1 month) and the similarity in structure to metanil yellow. Agarose-PAC hydrogels were used due to the prevalence of activated carbon as a water treatment method for removing PFAS from drinking water.

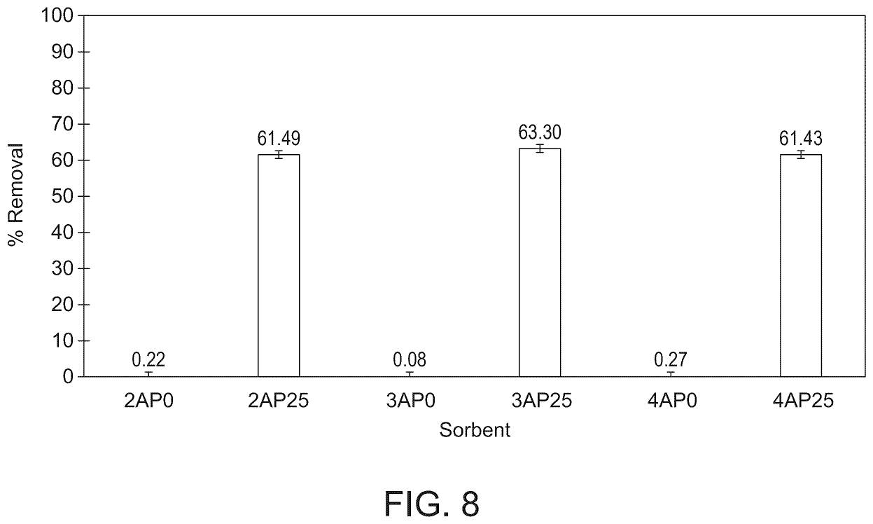

To evaluate the potential in vivo performance, thermally controlled sorption studies were conducted at a healthy body temperature of 37° C. The samples were left in solution for 90 hours, then the concentration of the supernatant was measured and removal was quantified below. The results indicate substantial yet incomplete sorption, indicating areas for improvement while showing promising potential for targeted sorption. The addition of cationic components could lead to improved sorption. See FIG. 8.

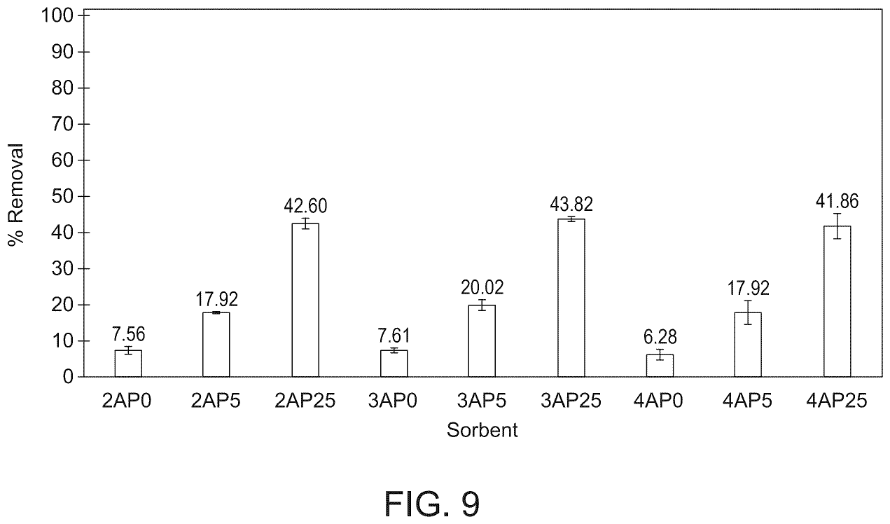

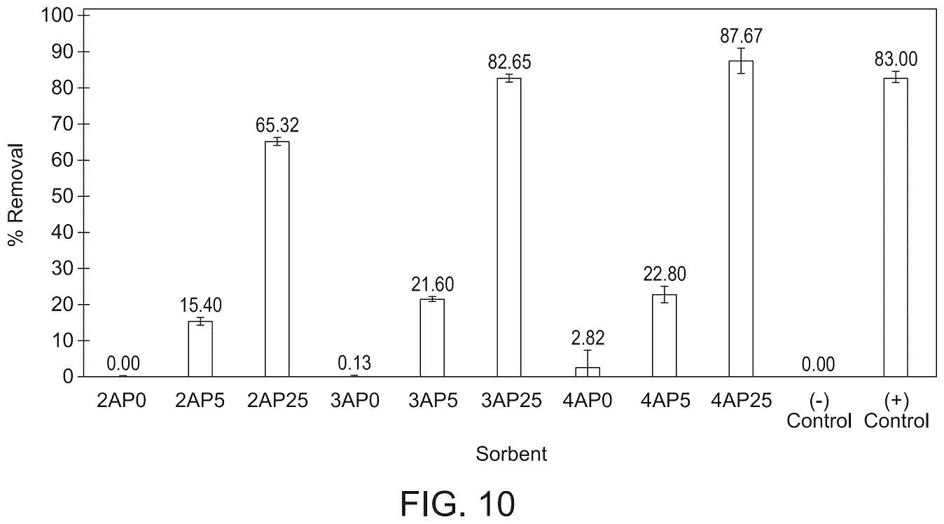

To determine both short-term and long-term sorption capabilities, agarose-PAC hydrogel systems with varying PAC concentrations were left in metanil yellow solution for 24 hours; then the supernatant concentration was measured, then the hydrogels were left for 2 weeks before the final data was collected. For the duration of the study (2 weeks), the hydrogels were in a temperature-controlled orbital shaker at a healthy body temperature of 37° C. The results of the short and long-term sorption indicate that significant sorption occurs within 24 hours; however, the sorption performance doubles at the two-week mark, indicating a gradual and prolonged sorption behavior. See FIGS. 9 and 10.

### CONCLUSIONS

Agarose-PAC hydrogels are good candidates for multipurpose sorption of various targets such as metanil yellow and tartrazine. Previous work also indicates that agarose-PAC hydrogels can also sorb indigo carmine. With these results, agarose-PAC is a viable potential candidate for the enterosorption of PFAS and synthetic food dyes.

Although the method and synthetic-based hydrogels and synthetic-based hydrogel composites of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A method for enterosorptive removal of a target molecule from a digestive tract of an individual, comprising orally administering an biopolymer-based hydrogel adapted to bind and remove the target molecule from the digestive track without being metabolized or absorbed into systemic circulation.

2. The method of claim 1, wherein the biopolymer-based hydrogel is a biopolymer-based hydrogel composite and the method further includes incorporating an additive into the biopolymer-based hydrogel selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

3. The method of claim 1, wherein the biopolymer-based hydrogel is an biopolymer-based hydrogel composite and the method further includes incorporating an additive into the biopolymer-based hydrogel selected from a list of additives consisting of an activated carbon, a powdered activated carbon, a granular activated carbon, an extruded activated carbon, an impregnated activated carbon, and combinations thereof.

4. The method of claim 1, wherein the biopolymer-based hydrogel is an biopolymer-based hydrogel composite and the method further includes incorporating an additive into the biopolymer-based hydrogel selected from a list of polymers consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, and combinations thereof.

5. The method of claim 1, wherein the biopolymer-based hydrogel is an biopolymer-based hydrogel composite and the method further includes incorporating an additive into the biopolymer-based hydrogel selected from a list of polymers consisting of poly(lysine), poly(ethyleneimine), chitosan, cationic cellulose, poly(acrylic acid), poly(methacrylic acid), and combinations thereof.

6. The method of claim 1, wherein the biopolymer-based hydrogel is an biopolymer-based hydrogel composite and the method further includes incorporating an additive into the biopolymer-based hydrogel selected from a list of additives consisting of a lectin, a DNA binding protein, an RNA binding protein, an albumin, a carbohydrate, and combinations thereof.

7. The method of claim 1, further including binding and removing per-fluoroalkyl substances from the digestive tract.

8. The method of claim 1, further including binding and removing poly-fluoroalkyl substances from the digestive tract.

9. The method of claim 1, further including binding and removing dye substances from the digestive tract.

10. The method of claim 1, further including binding and removing a synthetic food dye from the digestive tract.

11. The method of claim 1, further including administering the biopolymer-based hydrogel composite at a dosage rate of between 0.002 to 1.0 grams at a time interval of between every 4 hours and once a month.

12. The method of claim 2, further including a weight percentage of additive of between about 0.1% and 50% in the biopolymer-based hydrogel composite.

13. The method of claim 2, further including a weight percentage of additive of between about 0.1% and 20% in the biopolymer-based hydrogel composite.

14. A hydrogel system, comprising:
a biopolymer-based hydrogel adapted for oral administration to an individual wherein the biopolymer-based hydrogel is further adapted to bind and remove a target molecule from the digestive track without being metabolized or absorbed into systemic circulation.

15. The hydrogel system of claim 14, wherein the biopolymer-based hydrogel is an biopolymer-based hydrogel composite incorporating an additive into the biopolymer-based hydrogel selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

16. The hydrogel system of claim 14, wherein the biopolymer-based hydrogel is a biopolymer-based hydrogel composite and the method further includes incorporating an additive into the biopolymer-based hydrogel selected from a list of additives consisting of an activated carbon, a powdered activated carbon, a granular activated carbon, an extruded activated carbon, an impregnated activated carbon, and combinations thereof.

17. The hydrogel system of claim 14, wherein the biopolymer-based hydrogel is an biopolymer-based hydrogel composite and the method further includes incorporating an additive into the biopolymer-based hydrogel selected from a list of polymers consisting of poly(styrene), poly(methyl methacrylate), a poly(ethylene), polypropylene, poly(vinyl chloride), polytetrafluoromethylene, poly(dimethylsiloxane), polyester, polyurethane, poly(vinylidene fluoride), a fluoropolymer, and combinations thereof.

18. The hydrogel system of claim 14, wherein the biopolymer-based hydrogel is a biopolymer-based hydrogel composite and the method further includes incorporating an additive into the biopolymer-based hydrogel selected from a list of additives consisting of poly(lysine), poly(ethyleneimine), chitosan, cationic cellulose, poly(acrylic acid), poly(methacrylic acid), lectin, DNA binding protein, RNA binding protein, albumin, carbohydrate, and combinations thereof.

19. The hydrogel system of claim 15, further including a weight percentage of additive of between about 0.1% and 50% in the biopolymer-based hydrogel composite.

20. A method for enterosorptive removal of a target molecule from a digestive tract of an individual, comprising orally administering a hydrogel or hydrogel composite adapted to bind and remove the target molecule from the digestive track without being metabolized or absorbed into systemic circulation.
